(19) United States
(12) Patent Application Publication
(10) Pub. No.: US 2026/0074010 A1
(43) Pub. Date: Mar. 12, 2026

(54) METHOD FOR PRODUCING A COMPUTATIONAL REDUCTION VACCINE

(71) Applicant: Matthew Vernon Hanson, Sun Prairie, WI (US)

(72) Inventor: Matthew Vernon Hanson, Sun Prairie, WI (US)

(21) Appl. No.: 18/827,685

(22) Filed: Sep. 7, 2024

Related U.S. Application Data
(63) Continuation of application No. 17/138,065, filed on Dec. 30, 2020, now Pat. No. 12,116,599.

(51) Int. Cl. G16B 15/30; C12N 9/22; C12N 15/11; C12Q 1/02

(52) U.S. Cl. CPC G16B 15/30; C12N 9/22; C12N 15/111; C12Q 1/025; C12N 2310/20

(57) ABSTRACT

A system for the rapid development of vaccines or anti-bacterial drugs is required when working with pandemics. The easiest way to formulate these new vaccines is through computational reduction of existing organisms via statistical models. Once vaccine candidates are arrived at through this method, “Super Organisms” containing all of the computationally reducible fragments can then be taken through a Crispr reduction process wherein those computationally reducible fragments are removed. The result is a vaccine candidate which has possible problematic function partially or fully removed. The “neutered” version of the virus, as well as the DNA fragments and their mRNA transcriptions, can then be tested in a lab and in clinical trials for efficacy. This patent covers a vaccine candidate production methodology using computationally reducible fragments removed from Super Organisms either collectively or individually; as well as the DNA fragments themselves and the mRNA transcripts of those fragments.

### BACKGROUND OF THE INVENTION

The present invention is a “Continuation in Part” invention which finds its origins first in a provisional Patent Application #63/045,101 (the “101 Application”) filed Jun. 27, 2020, and its non-provisional follow-on Patent Application #17/138,065 (the “065 Application”).

It is important to note from the outset that the Specification language from both the 101 provisional and non-provisional 065 Applications are nearly identical, as discussed below.

This high level of similarity between the 101 and 065 Specifications (and Applications) was discussed in exhaustive detail in the examination of the 065 Application.

In addition, there are also two other precedential applications of note.

1) Provisional Application 63/045,121 (the “121 Application”) filed Jun. 28, 2020, was followed by Non-Provisional Patent Application #17/139,287 (the “287 Application”) filed Dec. 31, 2020; and 2) Non-Provisional Patent Application #17/137,209 (the “209 Application”) filed Dec. 29, 2020.

While these Applications are not relevant to the current instance, they do further establish a pattern of utilization of identical Specification language by this Applicant through five applications (two provisional, three non-provisional).

So the order of filing for all of those Applications was: 101, 121, 209, 065, 287. And the order of non-provisional examinations was: 287, 209, 065. Again, not relevant to the current instance but this does provide a bit of additional background for the reader.

The current instance 101 (provisional) and 065 (non-provisional) Applications were filed first and fourth respectively in that series.

These applications and examinations were highly contentious and not much fun for the Applicant, however, all three (287, 209, 065) were either granted (287 and 209) or are currently at the Notice of Allowance (065) stage.

This timeline sets the stage for the current application, though the only Applications relevant to the current instance are the instant 101 provisional and 065 non-provisional Applications.

The timeline, in other words, is useful as background, as there are several other “stops along the way” where the essentially the exact same Specification verbiage was used five times in sequence to describe a series of prototype “computational reduction vaccines”—a methodology patent for which is sought by this application.

It is also useful to keep in mind the timeline for these applications: Jun. 27, 2020 and Jun. 28, 2020 (the 101 and 121 provisional Applications respectively); and Dec. 29, 2020, Dec. 30, 2020, and Dec. 31, 2020 (the 209, 065, and 287 non-provisional Applications respectively). In other words, all five of these applications were filed over two very short time periods.

### BRIEF SUMMARY OF THE INVENTION

A “computational reduction vaccine” was introduced using very specific language.

In the examinations of the applications above, a “computational reduction vaccine” was described essentially as a kind of mathematical counting system, wherein mathematically significant sections of a virus could first be identified, then removed from a base organism to create a “neutered” version of that organism (a computational reduction), and then those same mathematically significant fragments could also be used in separate or complimentary DNA and mRNA compositions. There have been significant further refinements to the methodology in the interim which are not included here. The specific language in the 101 and 065 applications is quoted directly below and provides the basis for the claim(s) herein described.

### BRIEF DESCRIPTION OF THE DRAWINGS

FIG. 1. This drawing provides a simplified overview of the methodology, and is in line with Specification language, adding no further supplementary or complimentary material. A virus or bacteria at 1) is broken into fragments at 2) leading to two groups of fragments, mathematically significant DNA fragments at 3) and the mRNA transcription of those DNA fragments at 4). At 5) a “Super Organism” which contains all or most of the mathematically significant fragments is determined, after which a CRISPR or a similar tool is used to remove those fragments at 6) leading to a “computational reduction” organism at 7) wherein those statistically significant fragments have been removed from the virus or bacteria in question leaving a “neutered” version which can then be tested in a laboratory at 8) to see if problematic function remains; and the mathematically significant DNA fragments at 3) and their mRNA transcriptions at 4) can also be tested in the laboratory.

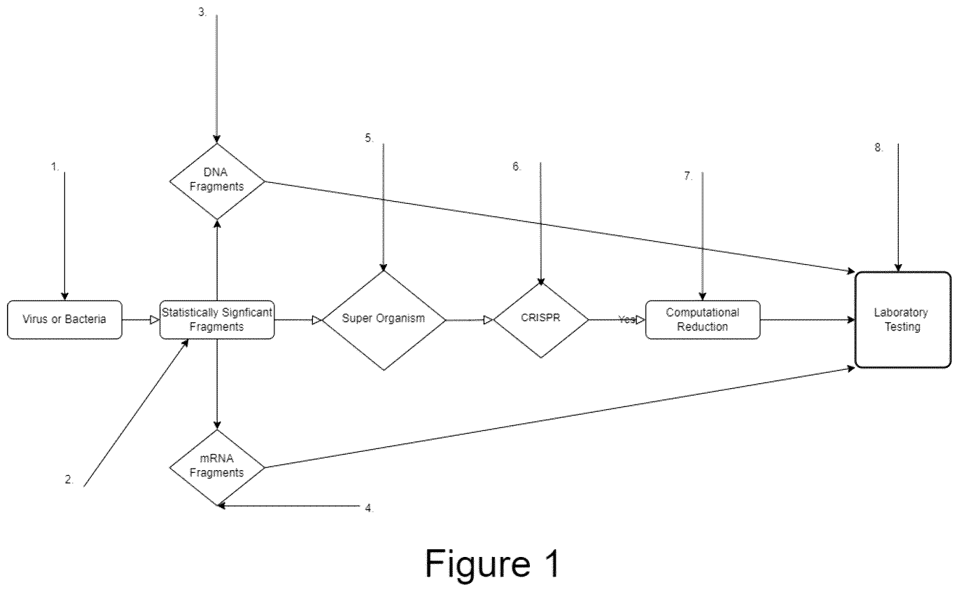

### DETAILED DESCRIPTION OF THE INVENTION

The specific language from the 101 Application is quoted directly below. As stated above, the 101 provisional Application was filed Jun. 27, 2020 and the 065 Application was filed on Dec. 30, 2020. For sake of convenience, the text below has been marked to show any difference between the 101 provisional and the 065 non-provisional language.

Cross-outs in the text are self-explanatory; any additions between the 101 and 065 Applications are underlined.

The reader should see that the verbiage is essentially identical, meaning that the 101 provisional Jun. 27, 2020 priority date should apply to the majority of this instant Application.

The relevant paragraphs from the 101 and 065 Applications which describe a computational reduction vaccine are [0002], [0004], [0005], [0009], [00010], [00012], and [00035].

The quoted paragraphs below are italicized for the reader's convenience, to set them apart from the actual numbered paragraphs of the present Application.

As stated above, the Applicant reserves the right to further reference the 101 and 065 Application specifications to strengthen his argument; in other words, while the text below is illustrative and conclusive, it might not be definitive.

The italicized paragraphs below include paragraph numbers from the 101 and 065 Applications for reference. The numbering systems (and paragraph positions) were identical in both Applications.

Again, the text below is the 101 Application original, marked with any 065 changes.

A computational reduction vaccine may be defined herein as a vaccine candidate which is arrived at by removing various non-repetitive fragments in a virus or bacteria first computationally, then via Crispr in (an actual) “Super-(Organism” or “Base) Organism” (an organism which contains all, or the majority, of those fragments), and then utilizing the remaining organism as a traditional “live” or “dead” vaccine, which even though marginally computationally reduced, is still recognizable by the human immune system as an invader and therefore provokes a useful immune response. That immune response then shields the recipient from the actual virus going forwards.

. . . the statistical analysis below will illustrate the efficacy of the method in determining the frequency of various structures, as well as the ability to track those structures through time. It is along those two lines—frequency of appearance, and consistency of appearance, across an entire genetic database that one can derive vaccine candidates computationally.

. . . it is more efficient to simply remove all potential problematic function fragments via various fragment length groups in order to create or two potential candidates instead of hundreds.

This invention introduces a new type of vaccine which is a computationally derived reductive vaccine. A computationally derived reductive vaccine utilizes statistical computation to arrive at a list of fragments which can then be removed from live viruses or bacteria via Crispr to arrive at “neutered” versions which can then form the basis for a vaccine.

Computational reduction in this case may be defined as non-laboratory computational reduction of organisms into fragments, which can then be assessed on the basis of frequency across an entire range of similar organisms as well as computationally tested to confirm that those structures are unique to the virus or bacteria in question.

When a “Super Organism” or Covid-19 sample which contains all, or most, of the fragments outlined below is found, that Super Organism can then be genetically modified in a laboratory using Crispr to remove those fragments. Once all of those fragments are removed from the organism, it can then be tested to see if problematic function remains. “Problematic function” in the case of Covid-19 is two-fold: functions of the virus which (have caused) cause high transmissibility rates, and functions of the virus which cause high mortality rates . . .. This patent provides a shortcut by simply removing all of the most likely candidates for those problematic functions by identifying fragments which appear often enough not to be considered mutation (i.e. fragments only appearing in one or two samples).

So to create a reductive vaccine, computationally those fragments are removed to create the vaccine candidate shown in this patent's sequence file.

So those are the relevant paragraphs.

Though as stated above the Applicant reserves the right to draw upon the 101 and 065 applications further in the revision of this Specification and/or Claims if required.

To summarize the paragraphs above, a method is described for the production of a new type of vaccine: a computationally derived reductive vaccine.

The approach involves a multi-step process, starting with computational analysis of viruses or bacteria to identify mathematically significant fragments.

These fragments are then computationally analyzed for their frequency and consistency across various genetic samples to determine which ones are likely to be crucial to an organism's functionality and/or potential pathogenicity.

The process involves using CRISPR or similar technology to edit a “Super-Organism”—a host organism containing all or most of the identified fragments.

By removing these fragments, it allows for the creation of “neutered” versions of an organism, which can in turn be tested to ensure it no longer possesses problematic functions, such as those causing high transmissibility or mortality rates in viruses like Covid-19.

The invention aims to streamline vaccine development by focusing on removing only those fragments most likely to be problematic, reducing the need to test thousands, or millions, of potential candidates.

The result, at least in theory, is a computational reduction vaccine candidate derived from a genetically modified organism that, despite being altered, remains recognizable by the human immune system and thus is capable of provoking a protective immune response while problematic function is either partially or fully disabled by the reduction itself.

The method offers a more efficient pathway for creating effective vaccines by leveraging computational tools and genetic engineering to target specific components of pathogens without necessarily needing to know the functionality of those components beforehand. In other words, the methodology provides the user with the ability to take significant shortcuts, save significant time, and make really good mathematically sound guesses about the probable location of problematic function.

The 101 and 065 Specifications clearly state this in no uncertain terms. The two precedent application GRANTS (the 287 and 209 Applications) and one Notice of Allowance (the 065 Application) means that the Applicant has successfully argued for practical application of the above methodology in prototype vaccine candidate design - not just once, but three times.

Further, the 101, 121, 287, 289, and 065 Applications all contain more or less exact copies of the Specification verbiage above. Again, that is not relevant here except in the current instance (the 101 and 065 Applications), but also illustrates that this wasn't a single “shot in the dark” application of the methodology; and that the methodology has, at the very least, theoretical merit in the eyes of at least five different Examiners.

The Examiner of the 065 Application took an “if it ain't in the Application, you can't use it” approach, but it should be clear to the reader at this point that the Specification of both the 101 and 065 Applications are nearly identical, those Specifications describe the methodology quite clearly, the 065 Application has been effectively granted a patent, and that as a result the single Claim here should be allowed.

The reason for this Continuation in Part application was that the Examiner of the 065 Application made a restriction for the application on Dec. 7, 2023, and the Applicant successfully traversed that restriction (election) requirement for this Application's Claim in a reply on Jan. 23, 2024.

Having herein described my invention, I claim:

1. A method for producing a computational reduction vaccine, comprising:
a. Computationally analyzing a virus or bacterium to identify mathematically significant fragments within the organism;
b. Determining the frequency and consistency of the identified fragments across a genetic database of similar organisms;
c. Selecting fragments for removal based on their mathematical significance and frequency;
d. Using CRISPR or a similar genetic engineering tool to remove the selected fragments from a “Super-Organism,” which is a host organism containing all or most of the identified fragments, thereby creating a “neutered” version of the organism;
e. Testing the “neutered” version of the organism to ensure it no longer possesses problematic functions, including but not limited to high transmissibility or mortality;
f. Utilizing the “neutered” organism as a basis for a vaccine candidate, wherein the vaccine provokes an immune response in a recipient while the problematic functions of the original pathogen are disabled by the reduction process;
g. Utilizing the DNA fragments or mRNA transcriptions of the DNA fragments identified in a) and b) as a basis for a vaccine candidate, wherein these fragments might be the actual problematic functions but are separated from other genetic machinery of the pathogen which might otherwise make those functions problematic;
h. The vaccine produced by the method of claim 1 being selected from a live attenuated vaccine, an inactivated vaccine, or a composition of mathematically significant DNA or mRNA transcripts.
